(19) United States
(12) Patent Application Publication
(10) Pub. No.: US 2019/0021690 A1
(43) Pub. Date: Jan. 24, 2019

(54) RADIOLOGY ASSEMBLY AND METHOD FOR ALIGNING SUCH AN ASSEMBLY

(71) Applicant: TRIXELL, Moirans (FR)

(72) Inventors: Bernard MUNIER, Moirans (FR); Guillaume JOLY, Moirans (FR); Fabien VERMONT, Meylan Cedex (FR); Patrick MENEROUD, Meylan Cedex (FR); Olivier SOSNICKI, Meylan Cedex (FR)

(73) Assignee: Trixell, Moirans (FR)

(21) Appl. No.: 15/778,593

(22) PCT Filed: Nov. 23, 2016
(86) PCT No.: PCT/EP2016/078563

§ 371 Date: May 23, 2018

(30) Foreign Application Priority Data
Nov. 23, 2015 (FR) 1561250

(51) Int. Cl. A61B 6/00; A61B 6/08

(52) U.S. Cl. CPC A61B 6/587; A61B 6/08

(57) ABSTRACT

A radiology assembly and a method for aligning such an assembly, comprising comprises: an x-ray tube for generating a beam of x-rays that is centered on a main emission direction; and substantially perpendicular to the main emission direction, a planar sensor to receive the x-rays. The radiology assembly comprises: a first divided emitter that is divided into two electromagnetic-field-emitting portions, the emitter being placed so as to emit a first electromagnetic field in a main direction that is substantially perpendicular to the main emission direction, each of the two emitting portions of the divided emitter being positioned on one side of the beam of x-rays; electromagnetic-field sensors that are securely fastened to the planar sensor and that are able to detect the electromagnetic fields emitted by the emitters and to generate a first electrical signal depending on the detected electromagnetic field; and a means for processing the first electrical signal to determine the relative position of the planar sensor with respect to the x-ray tube.

The invention relates to a radiology assembly and more precisely to the alignment of two elements of the radiology assembly, namely the planar sensor with respect to the x-ray tube. The invention also relates to a method for aligning such a radiology assembly. The invention pertains to the field of radiology (for example medical or veterinary radiology).

In the present patent application, the invention is presented in a case of application to a radiology assembly. Nevertheless, the invention may be applied in other fields requiring two elements to be correctly positioned with respect to each other.a

A radiology assembly consists of two elements: an x-ray tube and a planar sensor of radiographic images. The assembly is intended to mainly produce radiographic images of patients in a hospital setting. A patient, whom it is desired to radiograph, is placed between the x-ray tube and the planar sensor. The two elements must therefore be well-positioned, with respect to each other, so that all the x-rays emitted by the x-ray tube are captured by the planar sensor. The two elements are then said to be correctly aligned. The alignment must be carried out before the x-rays are emitted by the x-ray tube. The aim is to prevent the patient from being over-irradiated with x-rays that are not captured by the sensor.

Generally, the x-ray tube is aligned manually by an operator to face the planar sensor. The alignment is carried out translationwise and rotationwise. The alignment is generally carried out when the patient is in place, i.e. positioned between the x-ray tube and the planar sensor. There are many particular cases in which the planar sensor is masked. Mention may be made, by way of example, of the case in which the planar sensor is placed under a patient for a radiograph of the abdomen or of the pelvis. Mention may also be made of the case in which the planar sensor is placed under a sheet, under a stretcher or even in an incubator. It is therefore, in these cases, very difficult for the operator to align the x-ray tube with respect to the planar sensor.

Moreover, the environment of the planar sensor may be of several types. The environment may in particular be a hospital bed or a stretcher including metal frames or an incubator for premature babies. The environment of the sensor may therefore be an additional hindrance with regard to correct positioning of the x-ray tube with respect to the planar sensor.

Alignment of the first element with respect to the second element comprises correction of several defects: centering defect (the beam of x-rays is not centered on the planar sensor), orientation defect (the beam of x-rays is poorly oriented with respect to the plane of the planar sensor) and perpendicularity defect (the beam of x-rays does not strike the planar sensor perpendicularly). The perpendicularity defect is critical when an anti-scatter grid is used to produce the image. The grid is then placed on the planar sensor. The x-rays, in order to be able to be detected by the planar sensor, must strike the sensor perpendicularly to the planar sensor. The angular tolerance with respect to perpendicularity is small (only a few degrees).

There are several ways of proceeding with alignment of two elements. Mention may firstly be made of optical alignment in which the two elements are aligned by means of light beams that measure the relative position of one element with respect to the other. Optical alignment cannot be used in the field of radiology since the planar sensor is often partially masked by a sheet or by the patient.

Alignment may also be achieved by means of two beams of acoustic waves. However, as the alignment is carried out in the presence of the patient, the patient may mask all or some of the planar sensor. In addition, the presence of the patient may locally attenuate the acoustic waves and thus corrupt the measurement of the distance between the planar sensor and the x-ray tube.

It is also possible to carry out the alignment of two elements on the basis of a measurement of the propagation time of an electromagnetic wave. The measurement of the propagation time of a wave allows the distance between the two elements to be measured. By triangulation, it is possible to determine the relative position of the two elements with respect to each other. However, such an alignment technique cannot be successively employed in the case of an application to radiology since the propagation time of the electromagnetic wave may vary depending on the position of the patient between the two elements (x-ray tube and planar sensor). In addition, multiple echoes may be generated because of the environment (bed, stretcher, etc.), the echoes possibly having signal levels higher than the main signal.

On the same principle, an aligning technique exists based on the measurement of the attenuation of an electromagnetic signal to measure the distance between two elements. As, in the case of an application to radiology, the patient may locally attenuate the electromagnetic wave and therefore corrupt the measurement, this alignment is unsuitable.

Lastly a dental radiology system (patent FR 2 899 349) uses a plurality of electromagnetic-field emitters that are placed in the same plane and one or two electromagnetic-field receivers that are suitable for receiving the electromagnetic fields emitted by the emitters. The use of two receivers allows the angular orientation of the sensor to be determined, but gives no indication as to the angle of one element with respect to the other (generator tube with respect the planar sensor). In addition, the position of the emitters in a same plane gives only a mediocre indication as to the location of the planar sensor with respect to the generator tube. It will be noted that dental radiology covers a relatively short distance (20 to 30 cm) between the x-ray tube and the sensor, compared to the distance between the x-ray tube and the sensor in the field of medical radiology (rather about 1 to 2 m).

The invention aims to mitigate all or some of the aforementioned problems by providing a radiology assembly with a plurality of electromagnetic-field emitters that are securely fastened to the x-ray tube and that are positioned in separate planes, and with a plurality of electro-magnetic field sensors that are positioned on the planar sensor receiving the x-rays. This assembly allows the spatial position of the planar sensor to be unambiguously determined and therefore its position with respect to the x-ray tube to be determined.

To this end, one subject of the invention is a radiology assembly comprising:

an x-ray tube for generating a beam of x-rays that is centered on a main emission direction; and

substantially perpendicular to the main emission direction, a planar sensor that is intended to receive the x-rays;

characterized in that it comprises:
a first divided emitter that is divided into two electromagnetic-field-emitting portions, said emitter being placed so as to emit a first electromagnetic field in a main direction that is substantially perpendicular to the main emission direction, each of the two emitting portions of the divided emitter being positioned on one side of the beam of x-rays;
electromagnetic-field sensors that are securely fastened to the planar sensor and that are able to detect the electromagnetic field emitted by the first emitter and to generate an electrical signal depending on the detected electromagnetic field; and
a means for processing the first electrical signal, which is intended to determine the relative position of the planar sensor with respect to the x-ray tube depending on the first electrical signal.

According to one embodiment, the radiology assembly may furthermore comprise a so-called planar electromagnetic-field emitter, the so-called planar emitter being a coil composed of windings, the so-called planar emitter being placed so as to emit an electromagnetic field in a main direction that is substantially parallel to the main emission direction, the windings being passed through by the main emission direction.

According to another embodiment, the radiology assembly may comprise a second divided emitter that is divided into two electromagnetic-field-emitting portions, said emitter being placed so as to emit an electromagnetic field in a main direction that is substantially perpendicular to the main emission direction and that is secant to the main direction of the first electromagnetic field, each of the two emitting portions of the divided emitter being positioned on one side of the beam of x-rays. The sensors are able to detect the electromagnetic field emitted by the second divided emitter and to generate a second electrical signal depending on the detected electromagnetic field.

Advantageously, the processing means comprises means for distinguishing the generated electrical signals.

According to another embodiment, each of the two emitting portions of the divided emitter comprises a least one winding, and the main emission direction of the beam of x-rays is positioned between the at least one winding of the divided emitter.

According to another embodiment, the so-called planar emitter comprises at least one winding that is passed through by the main emission direction of the beam of x-rays.

The invention also relates to a method for aligning a radiology assembly according to the invention including the following steps:
Emission by the emitter of the electromagnetic field;
Detection of the electromagnetic field by the sensors;
Generation of an electrical signal by the sensors depending on the detected electromagnetic field; and
Determination of the relative position of the planar sensor with respect to the x-ray tube by the processing means depending on the electrical signal.

According to one embodiment, the aligning method according to the invention includes, beforehand, a calibration step intended to calibrate the electrical signal as a function of preset positions of the x-ray tube and of the planar sensor.

Advantageously, the step of emission by the at least one emitter of the at least one electromagnetic field includes the following steps:
Supplying the first divided emitter with a first electrical signal so that it emits a first electromagnetic field the main direction of which is substantially perpendicular to the main emission direction; and
Supplying the second divided emitter with a second electric field so that it emits a second electromagnetic field the main direction of which is substantially perpendicular to the main emission direction and secant to the main direction of the first electromagnetic field.

According to one embodiment, the step of emission by the at least one emitter of the at least one electromagnetic field includes the following step:

Supplying the so-called planar emitter with a third electrical signal so that it emits a third electromagnetic field the main direction of which is substantially parallel to the main emission direction.

Advantageously, the emitters are supplied with power at different times or simultaneously at different frequencies or simultaneously in phase offset so as to differentiate the electromagnetic fields emitted.

The invention will be better understood and other advantages will become apparent on reading the detailed description of one embodiment, which embodiment is given by way of example, the description being illustrated by the appended drawings, in which:

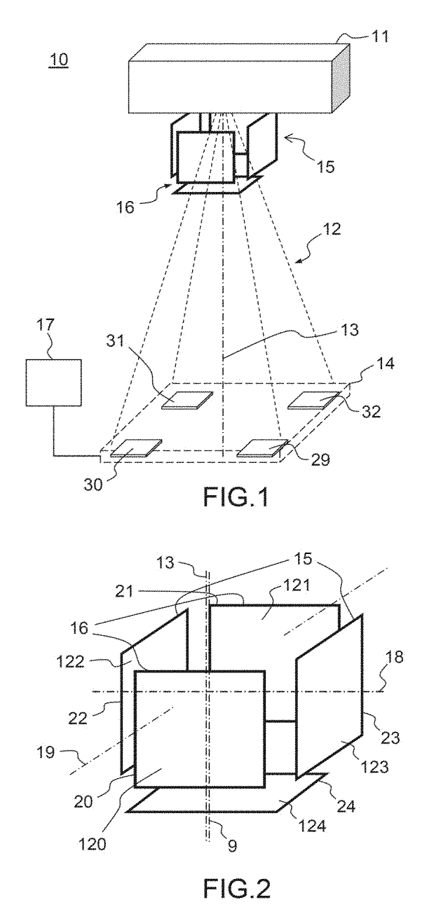

FIG. 1 shows one embodiment of a radiology assembly according to the invention;

FIG. 2 shows an example of an arrangement of the electromagnetic-field emitters according to the invention;

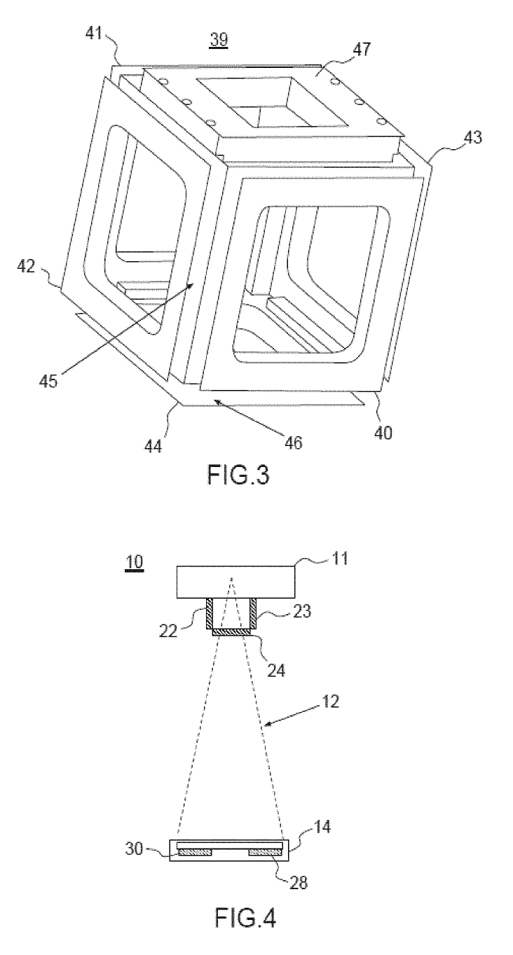

FIG. 3 shows an example of a holder of the electromagnetic-field emitters;

FIG. 4 shows a cross-sectional view of a radiology assembly according to the invention; and

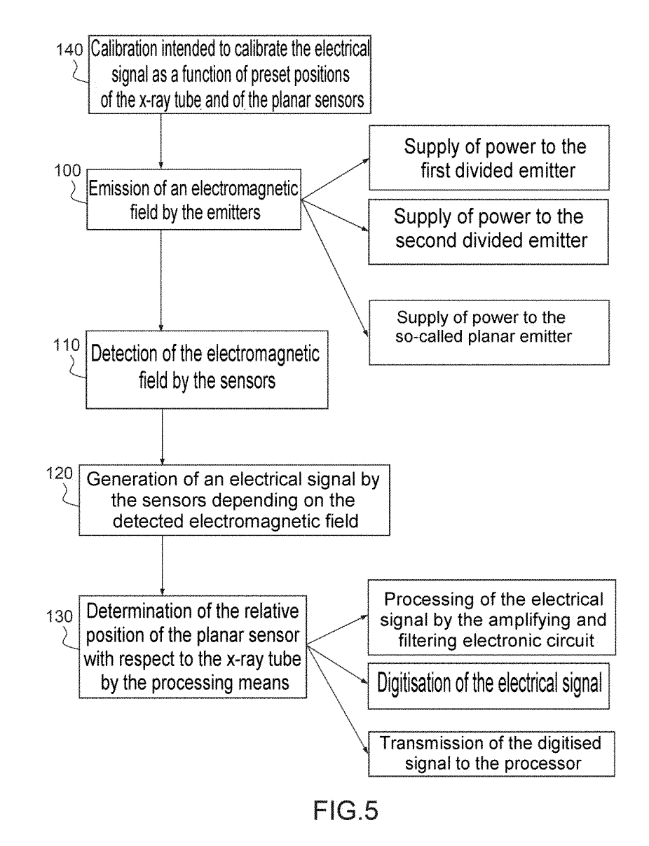

FIG. 5 schematically shows the steps of an aligning method according to the invention.

For the sake of clarity, elements that are the same have been given the same references in the various figures.

FIG. 1 shows one embodiment of a radiology assembly 10 according to the invention. The radiology assembly 10 comprises an x-ray tube 11 for generating a beam of x-rays 12 that is centered on a main emission direction 13. Substantially perpendicular to the main emission direction 13, the radiology assembly 10 comprises a planar sensor 14. The planar sensor 14 is intended to receive the x-rays 12. According to the invention, the radiology assembly comprises a first divided emitter 15 that is divided into two electromagnetic-field-emitting portions 20, 21 placed so as to emit a first electromagnetic field in a main direction that is substantially perpendicular to the main emission direction 13, each of the two emitting portions 20, 21 of the divided emitter 15 being positioned on one side of the beam of x-rays 12. Advantageously, the divided emitter 15 is securely fastened to the x-ray tube 11 for generating the beam of x-rays 12. In this configuration, the position of the x-ray tube 11 for generating the beam of x-rays 12 may be deduced from the main direction of the electromagnetic field emitted by the divided emitter 15.

Likewise, the radiology assembly may comprise a second divided emitter 16 made up of two electromagnetic-field-emitting portions 22, 23, said emitter being placed so as to emit an electromagnetic field in a main direction that is substantially perpendicular to the main emission direction 13 and secant to the main direction of the first electromagnetic field, each of the two emitting portions 22, 23 of the divided emitter 16 being positioned on one side of the beam of x-rays 12.

In other words, each divided emitter (15 for example) may be considered to be a pair of emitters (20, 21) the main faces of which are parallel to each other, each of the emitters being located on one side of the beam of x-rays. The pair of emitters 20, 21 (likewise for 22, 23) is equivalent to a virtual emitter located between the two emitters 20, 21, in the beam of x-rays. Considering one divided emitter (i.e. one pair of emitters), the emitted electromagnetic field is equivalent to the electromagnetic field emitted by the equivalent virtual emitter. This arrangement has the advantage of not obscuring the x-rays since the pair of emitters are located on either side of the beam of x-rays and not in the beam. Moreover, this arrangement of the emitters has the advantage of not damaging the emitters. Specifically, an equivalent emitter placed in the beam of the x-rays would be damaged by the x-rays during its use. In the case of our invention, the emitters are not subjected to the x-rays and are therefore preserved from the material resistance point of view.

The radiology assembly 10 also comprises electromagnetic-field sensors 29, 30, 31, 32 that are securely fastened to the planar sensor 14 and that are able to detect the electromagnetic fields emitted by the emitters 15, 16 and to generate an electrical signal depending on the detected electromagnetic fields. Generally, each of the electromagnetic-field sensors may comprise an amplifying and filtering electronic circuit intended to process the electrical signal generated by each of the sensors.

Lastly, the radiology assembly 10 comprises a means 17 for processing the electrical signal, which is intended to determine the relative position of the planar sensor with respect to the generator tube 11 depending on the electric signal generated by the sensors 29, 30, 31, 32.

FIG. 2 shows an example of an arrangement of the electromagnetic-field emitters 15 and 16 according to the invention. In FIG. 2, the radiology assembly comprises two divided emitters 15, 16 that are divided into two electromagnetic-field-emitting portions 20, 21 and 22, 23. The first divided emitter 15 that is divided into two electromagnetic-field-emitting portions 20, 21 is placed so as to emit a first electromagnetic field in a main direction 18 that is substantially perpendicular to the main emission direction 13. Each of the two emitting portions 20, 21 of the divided emitter 15 is positioned on one side of the beam of x-rays 12. Likewise, the second divided emitter 16 that is divided into two electromagnetic-field-emitting portions 22, 23 is placed so as to emit a first electromagnetic field in a main direction 19 that is substantially perpendicular to the main emission direction 13. Each of the two emitting portions 22, 23 of the divided emitters 16 is positioned on one side of the beam of x-rays 12.

An emitter may for example be a coil or a solenoid. An emitter consists of at least one winding through which a current may flow. If the surface represented by the winding of each of the emitting portions 20, 21 and 22, 23 is now considered, it will be noted that a surface 120 of the emitting portion 20 is substantially parallel to a surface 121 of the emitting portion 21. Furthermore, the electromagnetic field emitted by the divided emitter 15 has a main direction 18 that is perpendicular to the surfaces 120 and 121. On the same principle, a surface 122 of the emitting portion 22 is substantially parallel to a surface 123 of the emitting portion 23. Furthermore, the electromagnetic field emitted by the divided emitter 16 has a main direction 19 that is perpendicular to the surfaces 122 and 123. Advantageously, the surfaces 120 and 121 are perpendicular to the surfaces 122 and 123. In addition to being secant, the main directions 18 and 19 are then substantially perpendicular to each other. This arrangement is in particular advantageous if the x-ray tube 11 for generating the beam of x-rays 12 has an emission flux of square shape. Thus, the flux of x-rays 12 is emitted in the main emission direction 13, between the surfaces 120, 121, 122, 123, without intersecting the emitters 15, 16 (and therefore without damaging them) and without being obscured since the emitters 15, 16 are not located in the flux of x-rays 12. In other words, each of the two emitting portions 20, 21; 22, 23 of the divided emitter 15, 16 comprises at least one winding and the main emission direction 13 of the beam of x-rays 12 is positioned between the at least one winding of the divided emitter 15, 16.

Specifically, this arrangement allows it to be made so that each of the pairs of emitters 20, 21 and 22, 23, the respective surfaces 120, 121 and 122, 123 of which are parallel to each other (as already mentioned the surface 121 is substantially parallel to the surface 122, and likewise for the surfaces 122 and 123), is equivalent to a virtual emitter located at the center of the surfaces 120, 121, 122, 123 of the emitters 15, 16, level with the main emission direction 13 of the x-rays, whereas it would be impossible to place a single emitter at the center since the center is occupied by the beam of x-rays. Thus, the emitters may emit, in an off-centered position, an electromagnetic field equivalent to an electromagnetic field emitted in a centered position, without obscuring the x-rays emitted by the x-ray tube 11.

The radiology assembly 10 may furthermore comprise a so-called planar electromagnetic-field emitter 24 that is placed so as to emit an electromagnetic field in a main direction 9 that is substantially parallel to the main emission direction 13. A surface 124 of the emitter 24 is substantially perpendicular to the surfaces 120, 121, 122, 123. The so-called planar emitter 24 allows an electromagnetic field to be generated parallel to the main emission direction 13. The so-called planar emitter 24 possibly for example being a coil or a solenoid, it consists of at least one winding through which a current may flow. Furthermore, the flux of x-rays 12 may pass through the so-called planar emitter 24 level with the winding. In other words, the so-called planar emitter 24 comprises a least one winding that is passed through by the main emission direction 13 of the beam of x-rays 12. The flux of x-rays 12 is not obscured by the so-called planar emitter 24 because it passes therethrough through the one or more windings.

Arranging the emitters as shown in FIG. 2 allows electromagnetic fields the main directions of which are along three different axes that are perpendicular to one another to be obtained. Since the emitters are securely fastened to the x-ray tube 11 for generating the beam of x-rays 12, the electromagnetic fields along the three axes allow the position of the emitters with respect to the planar sensor 14 to be determined and therefore the position of the x-ray tube 11 for generating the beam of x-rays 12 to be determined with respect to the planar sensor 14. It will be noted that the three axes are not necessarily perpendicular to one another. The directions 18 and 19 may be secant and make any angle (therebetween and with the main emission direction 13). The relative position of the x-ray tube 11 with respect to the planar sensor 14 may also be determined.

In FIG. 2, the emitters are three in number (15, 16, 24, i.e. 4 emitting portions 20, 21, 22, 23 and one emitter 24) and positioned so as to form a rectangular parallelepiped. Nevertheless, it is entirely envisionable for there to be more than three emitters, each being positioned on one face of a polyhedron the number of faces of which would correspond to the number of emitting portions and emitters used. A larger number of emitters adds to the precision of the determination of the position of the planar sensor 14 with respect to the x-ray tube 11. Nevertheless, this larger number increases production costs and increases the complexity of the processing of the signals. Three emitters, as in FIG. 2, represents a very good compromise between the precision of the determination of the position of the planar sensor 14 with respect to the x-ray tube 11 and the complexity of the signal processing.

FIG. 3 shows an example of a holder 39 of the electromagnetic-field emitters. Corresponding to the configuration of FIG. 2, the holder 39 has faces 40, 41, 42, 43, 44 that are substantially perpendicular to one another. The face 42 comprises a groove 45 that is suitable for receiving the emitting portion 22. Likewise, the face 44 comprises a groove 46 suitable for receiving the emitter 24. The same goes for each of the faces. The holder 39 comprises an intermediate element 47, which is substantially perpendicular to the faces 40, 41, 42, 43 and substantially parallel to the face 44. The intermediate element 47 is a fastening means allowing the holder 39 (and therefore the emitters 15, 16, 24) to be securely fastened to the x-ray tube 11 for generating the beam of x-rays 12.

In the case of a configuration with a plurality of other emitters, the holder 39 then has another three-dimensional geometric shape with planar faces, each planar face having a groove arranged to house one emitter.

By virtue of the geometry presented in FIGS. 2 and 3, the pairs of surfaces that are parallel to each other (120 and 121, 122 and 123) and the placement of the emitters (for example a winding) in the grooves that are provided for this purpose, means that the left and right windings (of faces 42 and 43) and/or front and back windings (of faces 40 and 41) are very symmetric, allowing a magnetic field that is perfectly centered on the center of the geometric shape to be obtained without hindering the passage of the x-rays. It is not necessary to have a plurality of windings in the grooves of the lateral faces, the bottom winding, i.e. that of the face 44 in the groove 46, being sufficient for symmetry.

In other words, each divided emitter (15, 16) is divided into two electromagnetic-field-emitting portions (20, 21; 22, 23) that are configured to generate an electromagnetic field that is perfectly centered between the two faces that the emitting portions form. The two emitting portions each have a surface, the two surfaces being parallel to each other.

As shown in FIG. 1, the radiology assembly 10 comprises four electromagnetic-field sensors 29, 30, 31, 32. The four sensors 29, 30, 31, 32 may be integrated into the planar sensor 14. The sensors 29, 30, 31, 32 are intended to detect the electromagnetic fields emitted by the emitters 15, 16, 24 and to generate an electrical signal depending on the detected electromagnetic fields. It will be noted that the radiology assembly may comprise fewer than four or more than four electromagnetic-field sensors.

The sensors 29, 30, 31, 32 are integrated into the planar sensor 14. They are installed so that they do not disturb the acquisition of the radiological image. They are for example placed behind the detecting elements of the radiological image with respect to the entrance face of the x-rays. They may have any position on the planar sensor 14. In this case, a correction is required in order to determine the relative position of the x-ray tube 11 with respect to the planar sensor 14. If, in contrast, they have positions that are perfectly symmetric with respect to the center of the planar sensor, the x-ray beam 12 is perfectly centered with respect to the x-ray tube 11 when the sensors 29, 30, 21, 32 have a perfectly balanced signal.

The electromagnetic-field sensors 29, 30, 31, 32 may for example be coils, magnetometers, magnetoresistors, anisotropic magnetoresistors, magneto-transistors, magneto-diodes, fluxgates or Hall-effect sensors.

FIG. 4 shows a cross-sectional view of the radiology assembly 10 according to the invention. Each of the electromagnetic-field sensors 29, 30, 31, 32 may comprise an amplifying and filtering electronic circuit (not shown in the figure) that is intended to process the electrical signal generated by each of the sensors 29, 30, 31, 32.

The processing means 17 furthermore comprises a processor suitable for computing the relative position of the planar sensor 14 with respect to the x-ray tube 11.

Each sensor 29, 30, 31, 32 detects an electromagnetic field and generates an electrical signal depending on the amplitude of the detected electromagnetic field. The generated electrical signal is processed by the amplifying and filtering electronic circuit.

Depending on the type of sensor used, at any given time, each sensor 29, 30, 31, 32 may generate one or more pieces of information. If the sensor is single-axis, it generates a single piece of information. If the sensor is multi-axis, it generates a plurality of pieces of information. The use of multi-axis sensors allows the amplitude of the electromagnetic field and its orientation to be determined.

The detected signals are digitized and transmitted to the processor which processes the pieces of information in order to determine the relative position of the planar sensor 14 with respect to the x-ray tube 11 for generating the beam of x-rays. The pieces of information generated by the sensors 29, 30, 31, 32 are transmitted in digital form. They may be transmitted either over a wired link or over a wireless link.

For a given position of the planar sensor 14, the spatial position of the planar sensor 14 is determined from a set of pieces of information, in particular the pieces of information generated by each of the electromagnetic-field sensors 29, 30, 31, 32 when the emitter 15 is supplied with power, the pieces of information generated by each of the electromagnetic-field sensors 29, 30, 31, 32 when the emitter 16 is supplied with power and lastly the pieces of information generated by each of the electromagnetic-field sensors 29, 30, 31, 32 when the emitter 24 is supplied with power.

In our configuration, if the sensors are single-axis sensors, for a given position of the planar sensor 14, twelve pieces of information are generated. If the sensors are multi-axis sensors, then thirty six pieces of information are generated.

Computations using all of these pieces of information allow the spatial position of the planar sensor 14 to be unambiguously determined.

FIG. 5 schematically shows the steps of an aligning method according to the invention. The method for aligning a radiology assembly 10 according to the invention includes the following steps
Emission by an emitter 15, 16 or 24 of an electromagnetic field (step 100);
Detection of the electromagnetic field by the sensors 29, 30, 31, 32 (step 110);
Generation of an electrical signal by the sensors 29, 30, 31, 32 depending on the detected electromagnetic field (step 120); and
Determination of the relative position of the planar sensor 14 with respect to the x-ray tube 11 by the processing means 17 depending on the electrical signal (step 130).

In the case of a radiology assembly 10 comprising a plurality of emitters 15, 16, 24, the processing means 17 may comprise means for distinguishing the generated electrical signals. The method then includes the following steps:
Emission by the second emitter of an electromagnetic field (step 100);
Detection of the electromagnetic field by the sensors (step 110);
Generation of an electrical signal by the sensors 29, 30, 31, 32 depending on the detected electromagnetic field (step 120), said signal being distinct from the electrical signal generated by the sensors 29, 30, 31, 32 depending on the electromagnetic field emitted by the first emitter;
Determination of the relative position of the planar sensor 14 with respect to the x-ray tube 11 by the processing means 17 depending on the electrical signal (step 130).

The step 130 of determination of the relative position of the planar sensor with respect to the generator tube includes the following steps:
Processing of the electrical signal by the amplifying and filtering electronic circuit (step 131);
Digitization of the electrical signal (step 132); and
Transmission of the digitized signal to the processor (step 133).

The aligning method according to the invention may include, beforehand, a calibration step 140 that is intended to calibrate the electrical signal as a function of preset positions of the x-ray tube 11 and of the planar sensor 14. In this step, the relative position of the various elements (planar sensor 14 and x-ray tube 11) is recorded then used to determine correctional terms that will be taken into account in the step 130 of determination of the relative position of the planar sensor 14 with respect to the x-ray tube 11.

The method according to the invention therefore makes it possible to avoid over-irradiating the patient, who is placed between the x-ray tube 11 and the planar sensor 14, with x-rays that are not captured by the planar sensor 14.

The step 100 of emission by the at least one emitter 15, 16 of the at least one electromagnetic field includes the following steps:

supplying the first divided emitter 15 with power so that it emits a first electromagnetic field the main direction 18 of which is substantially perpendicular to the main emission direction 13; and

supplying the second divided emitter 16 with power so that it emits a second electromagnetic field the main direction 19 of which is substantially perpendicular to the main emission direction 13 and secant to the main direction 18 of the first electromagnetic field.

The step 100 of emission by the emitter 15, 16, 24 of then electromagnetic field may include the following step:

supplying the so-called planar emitter 24 with power so that it emits a third electromagnetic field the main direction 9 of which is substantially parallel to the main emission direction 13.

Lastly, the emitters 15, 16, 24 are supplied with the electrical signals at different times or simultaneously at different frequencies or simultaneously in phase offset so as to differentiate the emitted electromagnetic fields.

In other words, the divided first emitter 15 and the divided second emitter 16 may be supplied with power at different times or simultaneously at a different frequency or in phase offset. The fact of supplying the divided emitters with power at different times or simultaneously at a different frequency or in phase offset is one means for distinguishing the generated electrical signals.

Likewise, the so-called planar emitter 24 and the divided first emitter 15 and the divided second emitter 16 may be supplied with power at different times or simultaneously at different frequencies or in phase offset.

Generally, the emitters are supplied with power via AC signals in order to set them apart from permanent magnets and the Earth's magnetic field. The supply frequency is typically comprised between 100 Hz and 10 kHz.

The emitters are supplied with power at different times so that the sensors can easily separate the detected electromagnetic fields. In the case where the emitters are supplied with power simultaneously, the frequencies are different, and the choice of the frequencies is such that the signals output from the electromagnetic-field sensors may be easily separated.

When the aligning method is activated by an operator, the electro-magnetic field emitters 15, 16, 24 that are securely fastened to the x-ray tube 11 for generating the beam of x-rays 12 are activated via a digital link. The frequency of the electromagnetic field of each emitter is programmable. Each emitter may therefore emit a specific frequency that is distinct from the others. The electromagnetic-field sensors 29, 30, 31, 32 that are securely fastened to the planar sensor 14 receive the electromagnetic signal of the emitters 15, 16, 24. The level and frequency of the signal detected by each of the sensors are transmitted, over a digital link, to the processing means 17. The processing means 17 processes the data and delivers, to the operator, the information that he requires to manually align the x-ray tube 11 facing the planar sensor 14. The operator moves the x-ray tube 11 or the planar sensor 14 in order to optimize their alignment with respect to each other. The information allowing the alignment is for example delivered via a display screen that is integrated into the x-ray tube 11 or via another element connected to the radiology assembly. An audio signal, the frequency of which is modulated depending on the distance with respect to the optimal position, may also indicate to the user the precision of the alignment.

1. A radiology assembly comprising:
an x-ray tube for generating a beam of x-rays that is centered on a main emission direction; and
substantially perpendicular to the main emission direction, a planar sensor that is intended to receive the x-rays;
comprising:
a first divided emitter that is divided into two electromagnetic-field-emitting portions, said emitter being placed so as to emit a first electromagnetic field in a main direction that is substantially perpendicular to the main emission direction, each of the two emitting portions of the divided emitter being positioned on either side of the beam of x-rays;
electromagnetic-field sensors that are securely fastened to the planar sensor and that are able to detect the electromagnetic field emitted by the first emitter and to generate a first electrical signal depending on the detected electromagnetic field; and
a means for processing the first electrical signal, which is intended to determine the relative position of the planar sensor with respect to the x-ray tube depending on the first electrical signal.

2. The radiology assembly as claimed in claim 1, further comprising a planar electromagnetic-field emitter, the so-called planar emitter being a coil composed of windings, the planar emitter being placed so as to emit an electromagnetic field in a main direction that is substantially parallel to the main emission direction, the windings being passed through by the main emission direction.

3. The radiology assembly as claimed in claim 1, comprising a second divided emitter that is divided into two electromagnetic-field-emitting portions, said emitter being placed so as to emit an electromagnetic field in a main direction that is substantially perpendicular to the main emission direction and that is secant to the main direction of the first electromagnetic field, each of the two portions of the divided emitter being positioned on either side of the beam of x-rays, and wherein the sensors are able to detect the electromagnetic field emitted by the second divided emitter and to generate a second electrical signal depending on the detected electromagnetic field.

4. The radiology assembly as claimed in claim 3, wherein the processing means comprises means for distinguishing the generated electrical signals.

5. The radiology assembly as claimed in claim 1, wherein each of the two emitting portions of the divided emitter comprises a least one winding and in that the main emission direction of the beam of x-rays is positioned between the at least one winding of the divided emitter.

6. The radiology assembly as claimed in claim 2, wherein the planar emitter comprises at least one winding that is passed through by the main emission direction of the beam of x-rays.

7. A method for aligning a radiology assembly as claimed in claim 1, comprising the following steps:
emission by the emitter of the electromagnetic field;
detection of the electromagnetic field by the sensors;
generation of the electrical signal by the sensors depending on the detected electromagnetic field; and
determination of the relative position of the planar sensor with respect to the x-ray tube by the processing means depending on the electrical signal.

8. The aligning method as claimed in claim 7, comprising, beforehand, a calibration step intended to calibrate the electrical signal as a function of preset positions of the x-ray tube and of the planar sensor.

9. The aligning method as claimed in claim 7, wherein the radiology assembly comprises a second divided emitter that is divided into two electromagnetic-field-emitting portions, said emitter being placed so as to emit an electromagnetic field in a main direction that is substantially perpendicular to the main emission direction and that is secant to the main direction of the first electromagnetic field, each of the two portions of the divided emitter being positioned on one side of the beam of x-rays, and wherein the sensors are able to detect the electromagnetic field emitted by the second divided emitter and to generate a second electrical signal depending on the detected electromagnetic field, the method further comprising the following steps:
emission by the second emitter of the electromagnetic field;
detection of the electromagnetic field by the sensors;
generation of the second electrical signal by the sensors depending on the detected electromagnetic field, this signal being distinct from the first electrical signal generated by the sensors; and
determination of the relative position of the planar sensor with respect to the x-ray tube by the processing means depending on the first and second electrical signals.

10. The aligning method as claimed in claim 7, wherein the radiology assembly comprises a planar electromagnetic-field emitter, the so-called planar emitter being a coil composed of windings, the planar emitter being placed so as to emit an electromagnetic field in a main direction that is substantially parallel to the main emission direction, the windings being passed through by the main emission direction the method further comprising the following steps:
emission by the planar emitter of the electromagnetic field;
detection of the electromagnetic field by the sensors;
generation of a third electrical signal by the sensors depending on the detected electromagnetic field, this signal being distinct from the first electrical signal generated by the sensors; and
determination of the relative position of the planar sensor with respect to the x-ray tube by the processing means depending on the first and third electrical signals.

11. The aligning method as claimed in claim 9, wherein the emission by the emitters of the electromagnetic fields includes a step of supplying the emitters with power, and wherein the emitters are supplied with power at different times or simultaneously at different frequencies or simultaneously in phase offset so as to differentiate the electromagnetic fields emitted.
